(19) United States
(12) Patent Application Publication
(10) Pub. No.: US 2021/0386668 A1
(43) Pub. Date: Dec. 16, 2021

(54) Cyclodextrin for use in the treatment and prevention of late phase bronchoconstriction in allergen-induced asthma

(71) Applicant: Universite de Liege, Liege (BE)

(72) Inventors: Didier Cataldo, Olne (BE); Brigitte Evrard, Embourg (BE)

(21) Appl. No.: 17/347,683

(22) Filed: Jun. 15, 2021

(30) Foreign Application Priority Data
Jun. 15, 2020 (BE) BE2020/5430

(51) Int. Cl. A61K 9/00; A61K 9/08; A61K 31/724; A61P 37/08

(52) U.S. Cl. CPC A61K 9/0078; A61P 37/08; A61K 31/724; A61K 9/08

(57) ABSTRACT

An inhalable cyclodextrin for use in the treatment and prevention of late phase bronchoconstriction in allergen-induced asthma is disclosed. Further disclosed is the use of a cyclodextrin in the treatment and prevention by inhalation of late phase bronchoconstriction in allergen-induced asthma.

### TECHNICAL FIELD

Asthma is a complex and multifactorial disease characterized by chronic airways inflammation affecting more than 300 million individuals worldwide. Asthma results in the constriction of airways, so called bronchoconstriction. Two forms of asthma are distinguished, non-allergen-induced and allergen-induced asthma.

### BACKGROUND OF THE INVENTION

Asthma

In allergen-induced asthma, an inflammatory response is generated by antigens. This response involves different cell types from innate and adaptive immune systems. These cells recruit and activate inflammatory cells leading to bronchial hyper-reactivity, mucus overproduction and airway wall remodeling. Membrane lipid microdomains regulate cellular signaling cascades. These include complex lipid-protein interactions and clustering of specific receptors. Cholesterol is a key component of liquid-ordered domains on cell membrane referred to as lipid rafts. Lipid rafts are also involved in allergen presentation and subsequent T-cell activation through localized enrichment of MHC class II molecules at the surface of antigen presenting cells.

Early Phase Reaction in Allergen-Induced Asthma

The early-phase bronchoconstriction usually happens immediately after allergen-exposure. Mast cells produce mediators that cause changes in the airways. Some mediators immediately cause inflammation in the early phase.

Late Phase Reaction in Allergen-Induced Asthma

Late phase reaction around occurs two hours to four hours after initial exposure to an antigen. Mediators induce chemotactic recruitment and activation of eosinophils and neutrophils during the late-phase reaction. The reinforcements cause persistent airway inflammation. This makes airways increasingly hypersensitive to asthma triggers and increases the risk for future asthma attacks. The late phase may last 12 hours to 24 hours.

Cyclodextrin for Use in Treatment of Pulmonary Disorders

Cyclodextrins have been proposed for use in the treatment of pulmonary disorders including asthma:

EP1799231 to the University of Liege discloses the use of a cyclodextrin compound for the manufacturing of a medicament for the treatment and prevention of bronchial inflammatory diseases, particularly for asthma.

EP2900246A1 to SolAeromed discloses the use of methyl-beta-cyclodextrin for the treatment of pulmonary surfactant dysfunction. This publication claims that oxidative damage to pulmonary surfactant arises due to the interaction between reactive oxygen species and cholesterol. It further claims that methyl-beta-cyclodextrin may restore normal function to dysfunctional surfactant removed from the lungs of children with cystic fibrosis and non-cystic fibrosis bronchiolitis.

US2010173869A1 to SolAeromed discloses a method for treatment of a surfactant, in particular a pulmonary surfactant. The surfactant is treated with a lipid sequestrating or cholesterol sequestrating surfactant treatment agent, in which given, in particular neutral lipids or cholesterol are selectively sequestrated by means of the surfactant treatment agent, such that the effect of the lipids and/or the effect of the cholesterol on the surfactant is reduced or reversed.

US2013029937A1 to SolAeromed discloses a method for enhancing a surfactant through cyclodextrin. The document relates to a method of mitigating oxidative damage to pulmonary surfactant by adding cyclodextrin as a cholesterol-sequestrating agent. This publication further discloses a method for treating a patient having surfactant dysfunction due to oxidative damage to pulmonary surfactant by administering a surfactant-protective amount of a cyclodextrin as a cholesterol-sequestrating agent to protect the surfactant from the negative effects of oxidative degradation.

Finally, EP3151836A1 to the University of Liège and Paul Maes discloses pharmaceutical compositions formulated with a cyclodextrin compound, in particular HPBCD and a budesonide derivative for the treatment and/or prevention of pulmonary inflammatory disease.

However, none of the above publications discloses the crucial difference between the treatment of early and late phase of allergen-induced asthma.

Thus, there is still an urgent need to further improve the efficacy of cyclodextrins in the treatment and prevention of allergen-induced asthma.

The present inventors now have surprisingly found that cyclodextrin may be used in the treatment of late phase bronchoconstriction in mild to moderate asthmatics. The applicants suggest that cyclodextrins lead to a perturbation of lung T-cell membranes organization that impair their activation after allergen recognition.

### SHORT DESCRIPTION OF THE INVENTION

A first aspect of the invention is an inhalable cyclodextrin or a pharmaceutically acceptable derivative thereof for use in the treatment or prevention of late phase bronchoconstriction in allergen-induced asthma.

In another aspect, the cyclodextrin is Hydroxypropyl-beta-cyclodextrin.

In another aspect, the cyclodextrin is an inhalable aqueous solution.

In another aspect, the cyclodextrin concentration is from 5 millimolar to 50 millimolar.

In another aspect, the cyclodextrin concentration is from 7 millimolar to 40 millimolar.

In another aspect, the cyclodextrin concentration is from 10 millimolar to 30 millimolar.

In another aspect, the cyclodextrin is a spray-dried powder.

In another aspect, the cyclodextrin is administered in an amount effective to reduce membrane order in cells.

In another aspect, the cyclodextrin is administered per inhalation in the amount of 0.1 mg to 30 mg per day.

In another aspect, the cyclodextrin is administered per inhalation in the amount of 0.5 mg to 20 mg per day.

In another aspect, the cyclodextrin is administered per inhalation in the amount of 1 mg to 10 mg per day.

In another aspect, the cyclodextrin is administered to children aged up to two years per inhalation in the amount of 0.1 mg to 0.5 mg per day.

In another aspect, the cyclodextrin is administered to children aged from two to 6 years per inhalation in the amount of 0.5 mg to 1 mg per day.

In another aspect, the cyclodextrin is administered to children aged from 6 years to 14 years per inhalation in the amount of 1 mg to 2 mg per day.

In another aspect, the cyclodextrin is administered from 0.1 mg to 15 mg per day in mild to moderate allergen-induced asthma and from 1 mg to 30 mg in severe allergen-induced asthma.

Another aspect of the invention is a method of treatment of T-cell dysfunction in pulmonary tissue, wherein cyclodextrin is administered per inhalation in an amount effective to reduce membrane order in the cells in subjects with T-cell dysfunction in their pulmonary tissue, preferably without causing treatment limiting side effects, such as those selected from the group consisting of renal clearance, hepatic impairment as expressed by elevated levels of transaminase, and wheezing after administration, as compared to subjects untreated with the cyclodextrin.

In another aspect of the method of treatment of T-cell dysfunction, the cyclodextrin is administered per inhalation in the form of a 15 mM HPBCD saline isotonic solution.

In another aspect of the method of treatment of T-cell dysfunction, the cyclodextrin is administered per inhalation in the form of a 15 mMol HPBCD PBS ph7.4 based solution.

In another aspect of the method of treatment of T-cell dysfunction, the cyclodextrin is administered per inhalation in the form of a 5 mMol HPBCD saline isotonic solution.

In another aspect of the method of treatment of T-cell dysfunction, the cyclodextrin is administered per inhalation in the form of a 25 mMol HPBCD PBS ph7.4 based solution.

In another aspect of the method of treatment of T-cell dysfunction, the cyclodextrin is administered per inhalation in the form of a 40 mMol HPBCD saline isotonic solution.

In another aspect of the method of treatment of T-cell dysfunction, the cyclodextrin is administered per inhalation in the form of a 25 mMol HPBCD saline isotonic solution.

In another aspect of the method of treatment of T-cell dysfunction, the cyclodextrin is administered per inhalation in the form of a 10 mMol HPBCD citrate ph. 4.5 based solution.

In another aspect of the method of treatment of T-cell dysfunction, the cyclodextrin is administered per inhalation in the form of a 40 mMol HPBCD citrate ph. 4.5 based solution.

In another aspect of the method of treatment of T-cell dysfunction, the cyclodextrin is administered per inhalation in the form of a 50 mMol HPBCD saline isotonic solution.

The cyclodextrins may be administered in an isotonic solution or a hypertonic solution.

A solution is isotonic when its effective osmole concentration is the same as that of the cytosol inside the cell and in particular the respiratory, preferably the pulmonary mucosa cells.

A hypertonic solution is called hypertonic if it has a greater concentration of solutes than the cytosol inside the cell and in particular the respiratory, preferably the pulmonary mucosa cells.

It is further preferred that the pH of the composition is adjusted to 3.5 to 7.5, preferably from 6.5 to 7.

In order to adjust the pH, surface tension, viscosity, osmolality, stability, taste and other properties of the composition, one or more further excipients may be used. For example, the composition may comprise one 25 or more excipients selected from pharmaceutically acceptable organic acids, salts of organic acids, inorganic acids, inorganic salts, bases, sugars, sugar alcohols, stabilizers, antioxidants, surfactants, preservatives, and taste masking agents.

### DETAILED DESCRIPTION OF THE INVENTION

The present inventors have surprisingly found that cyclodextrin may be used to extract lipids or reduce membrane order in the membrane of epithelial cells and in particular in the membrane of T-cells in lung parenchyma. The reduction in membrane order through cyclodextrin inhalation leads to decreased T-cell activation and T-cell proliferation. T-cell activation and T-cell proliferation are impacting the late phase bronchoconstriction in allergen-induced asthma. Thus, a first aspect of the invention is an inhalable cyclodextrin or a pharmaceutically acceptable cyclic derivative thereof for use in the treatment of late phase bronchoconstriction in allergen-induced asthma.

### Definitions

The term “asthma” describes a disease resulting in chronic inflammation and constriction of the airways.

The term “bronchoconstriction” means the constriction of the airways in the lungs due to the tightening of surrounding smooth muscle, with consequent coughing, wheezing, and shortness of breath due to an immunological reaction involving release of inflammatory mediators. In one embodiment bronchoconstriction is measured by a decrease in FEV1.

The term “FEV1” describes the forced expiratory volume in 1 second. FEV1 is the volume of air that can forcibly be blown out in first second after full inspiration.

The term “early phase” means bronchoconstriction that usually occurs immediately after allergen-exposure in allergen-induced asthma up to 60 minutes after allergen exposure.

The term “late phase” bronchoconstriction describes bronchoconstriction from 180 minutes to 360 minutes from allergen-exposure. An example of late phase bronchoconstriction is a decrease in FEV1 of 15% from 180 minutes to 360 minutes from allergen-exposure. In some embodiments the late phase bronchoconstriction is lasting several hours, for example from 180 minutes to five, six, seven, eight, nine or ten hours from allergen-exposure. In some embodiments the late phase lasts up to 24 hours from allergen-exposure.

The term “membrane disorder” or “reduction of membrane order” means a reduction in organization and rigidity of cell membranes, in particular T-cell membranes of lung parenchyma. In one embodiment disorder means an increase in mobility and polarity of phospholipids in T-cell membranes. In one embodiment, mobility and polarity of phospholipids are measured through labelling with fluorescents such as Laurdan or through blue staining of lung sections. The lipid dynamic and fluidity at the acyl chain after cyclodextrin incubation may be measured at 37 degree Celsius. Anisotropy at 37 degree Celsius may be used to measure membrane rigidity. An exemplary embodiment of a way to measure membrane disorder or reduction of membrane order is given in example 1 below.

The term “T-cell proliferation” means an increase in T-cells over a given period of time. In one embodiment, T-cell proliferation is measured by flow cytometry analysis of lung TH2 cells and total leukocyte counts. An exemplary embodiment of a way to measure T-cell proliferation is given in example 1 below. In one embodiment, naïve CD4+ T cells are exposed to HPBCD at a concentration of 5 mM and an anti-CD3 (3 mcg/ml) for 24 hours or 48 hours with HPBCD (5 mM) or with culture medium alone at 37° C. 5% CO2. During the last 2 hours of the proliferation test, Bromodeoxyuridine is added to the medium and incorporation is quantified by ELISA. IL-2 secretion is measured in the medium after 24 hours and 48 hours of anti-CD3 stimulation through ELISA.

The term “treatment” or “treat” describes inhalation of a cyclodextrin to reduce late phase bronchoconstriction in asthma. In particular, the term “treatment” describes inhalation of a cyclodextrin to reduce T-cell membrane order.

The term “prevention” describes any reduction of the risk of late phase bronchoconstriction in asthma by inhalation of a cyclodextrin. In particular, the term “prevention” describes inhalation of a cyclodextrin to reduce T-cell membrane order.

The terms “effective amount” or “therapeutically effective amount,” as used herein, refer to an amount of an active agent as described herein that is sufficient to achieve, or contribute towards achieving, one or more desirable clinical outcomes, such as those described in the “treatment” description above. An appropriate “effective” amount in any individual case may be determined using standard techniques known in the art, such as a dose escalation study. In some embodiments, as used herein, the term “therapeutically effective amount” is meant to refer to an amount of an active agent or combination of agents effective to ameliorate, delay, or prevent the symptoms.

The term “cyclodextrin” describes oligosaccharides composed of glucopyranose units. The major unsubstituted cyclodextrins are usually prepared by the enzymatic degradation of starch. The cyclodextrin of the invention may be any cyclodextrin, in particular alpha-, beta- and gamma-cyclodextrins, comprising 6, 7 and 8 glucopyranose units, respectively. In another embodiment of the invention derivatives of cyclodextrins are used, for example chemically modified cyclodextrins, which may have increased water solubility over unmodified cyclodextrins. Examples of such derivatives include in particular 2-hydroxypropyl-beta-cyclodextrin (HPBCD), 2-hydroxypropyl-gamma-cyclodextrin (HPGCD), sulfobutylether-beta-cyclodextrin (SBEBCD), and methyl-beta-cyclodextrin (MBCD).

The term “pharmaceutically acceptable derivative” of a cyclodextrin describes cyclic organic compounds derived of cyclodextrins that are able to create epithelial membrane disorder in lung parenchyma to an extent comparable to cyclodextrins.

The term “aqueous solution” as used herein refers to a composition comprising at least one cyclodextrin, water and optionally one or more other components suitable for use in pharmaceutical delivery such as carriers, stabilizers, diluents, dispersing agents, suspending agents, thickening agents, excipients, and the like. In some embodiments, the pharmaceutical composition is free of alpha or gamma-cyclodextrin.

The term “active pharmaceutical ingredient” refers to any substance or combination of substances used in a finished pharmaceutical product, intended to furnish pharmacological activity or to otherwise have direct effect in the diagnosis, cure, mitigation, treatment or prevention of disease, or to have direct effect in restoring, correcting or modifying physiological functions in human beings. Preferably, the term “active pharmaceutical ingredient” refers to a molecule that is intended to be biologically active, for example for the purpose of treating inflammatory, autoimmune, or pulmonary disease, disorder, or condition.

Reduction in Membrane Order of T-Cells

The present inventors have surprisingly found that cyclodextrin may be used to extract lipids or reduce membrane order in the membrane of epithelial cells and in particular in the membrane of T-cells in lung parenchyma. The reduction in membrane order through cyclodextrin inhalation leads to decreased T-cell activation and T-cell proliferation. T-cell activation and T-cell proliferation are impacting the late phase bronchoconstriction in allergen-induced asthma. Thus, a first aspect of the invention is an inhalable cyclodextrin or a pharmaceutically acceptable cyclic derivative thereof for use in the treatment of late phase bronchoconstriction in allergen-induced asthma. The inventors further show that cyclodextrins given by inhalation decrease allergen-induced inflammation and associated hyperresponsiveness in allergen-induced bronchoconstriction.

In another embodiment, the cyclodextrin of the invention is used to reduce T-cell proliferation or T-cell activation.

Also, the present inventors have found that cyclodextrins may have no impact on early phase asthma.

In a preferred embodiment, the cyclodextrin of the invention is Hydroxypropyl-beta-cyclodextrin.

In another aspect, the asthma is mild to moderate allergen-induced asthma.

Another aspect of the invention is a composition comprising a cyclodextrin or a pharmaceutically acceptable cyclic derivative thereof for use in the treatment of late phase bronchoconstriction in allergen-induced asthma.

Amount, Concentration and Dosage

The cyclodextrin of the present invention is used to treat or prevent the late phase bronchoconstriction of allergen-induced asthma. In one embodiment, it is administered to patients that show a late phase bronchoconstriction in allergen-induced asthma. It is administered in an amount effective to reduce the bronchoconstriction in late phase asthma as compared to placebo patients.

In one embodiment, cyclodextrin is administered in a daily dose from 0.1 mg to 30 mg, preferably from 0.5 mg to 20 mg, from about 1 mg to 10 mg, even more preferably from about 5 mg to 10 mg.

In another embodiment, the cyclodextrin is administered to children aged up to two years per inhalation in the amount of 0.1 mg to 0.5 mg per day.

In one embodiment, the cyclodextrin is administered to children aged from two to 6 years per inhalation in the amount of 0.5 mg to 1 mg per day.

In one embodiment, the cyclodextrin is administered to children aged from 6 years to 14 years per inhalation in the amount of 1 mg to 2 mg per day.

In one embodiment, the cyclodextrin is administered from 0.1 mg to 15 mg per day in mild to moderate allergen-induced asthma and from 1 mg to 30 mg in severe allergen-induced asthma.

In one embodiment, the cyclodextrin is administered once per day per inhalation.

In another embodiment, the cyclodextrin is administered twice per day per inhalation, preferably once in the morning and once in the evening.

In another embodiment, the cyclodextrin is administered three times per day per inhalation, preferably once in the morning, once at noon and once in the evening.

However, more inhalations may be foreseen per day, for example four, five, six or seven times per day.

In one embodiment, the cyclodextrin is a liquid composition comprising cyclodextrin in the range from 1 mg/ml to 100 mg/ml, preferably from 5 mg/ml to about 50 mg/ml, and more preferably from 10 mg/ml to about 30 mg/ml. Other preferred concentrations range from 15 mg/ml to 25 mg/ml.

In another embodiment, the cyclodextrin concentration in the liquid composition is from one millimolar to 100 millimolar, preferably three millimolar to 80 millimolar, even more preferably 5 millimolar to 50 millimolar, even more preferably 7 millimolar to 40 millimolar, even more preferably 10 millimolar to 30 millimolar, and even more preferably 12.5 millimolar to 17.5 millimolar.

Further Ingredients

In one embodiment, the cyclodextrin is associated with a further active pharmaceutical ingredient.

In another embodiment, no further active pharmaceutical ingredient is associated with the cyclodextrin of the present invention.

In a further embodiment of the invention, the inhalable composition further comprises a component selected from the group consisting of carriers, stabilizers, diluents, dispersing agents, suspending agents, thickening agents, excipients, and antimicrobial preservatives. In aqueous solutions, these components are present in low amounts, typically in the range of 0.1 mg/ml to 5 mg/ml.

Another aspect of the invention is the use of a cyclodextrin or a composition of the invention in the treatment and prevention by inhalation of late phase bronchoconstriction in allergen-induced asthma.

Another aspect of the invention is an aerosol generating device or dry powder inhaler comprising the cyclodextrin or the composition of the invention.

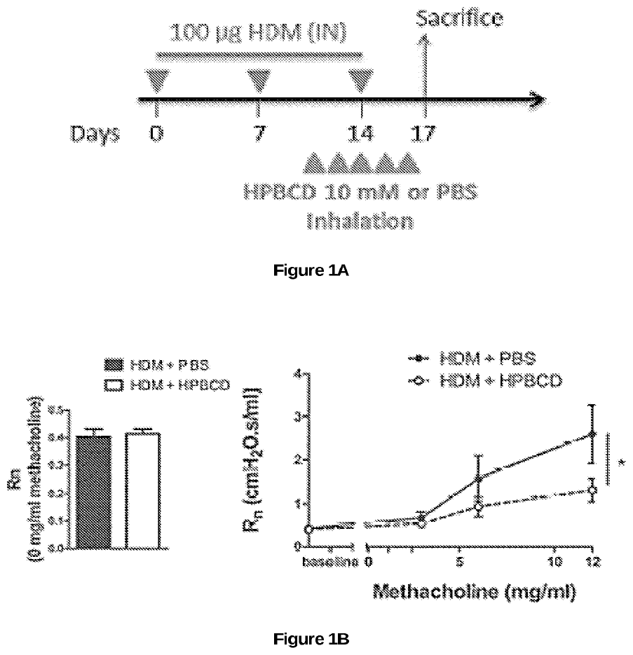

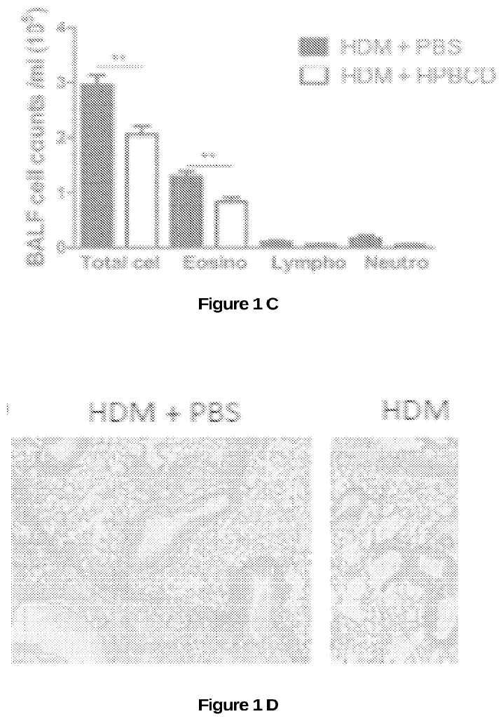

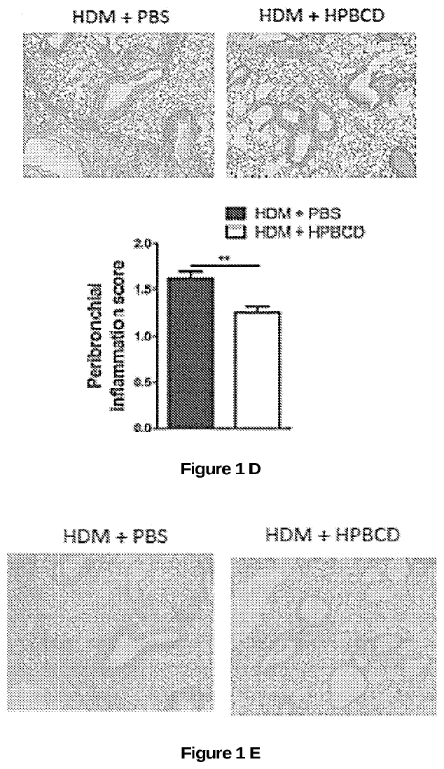

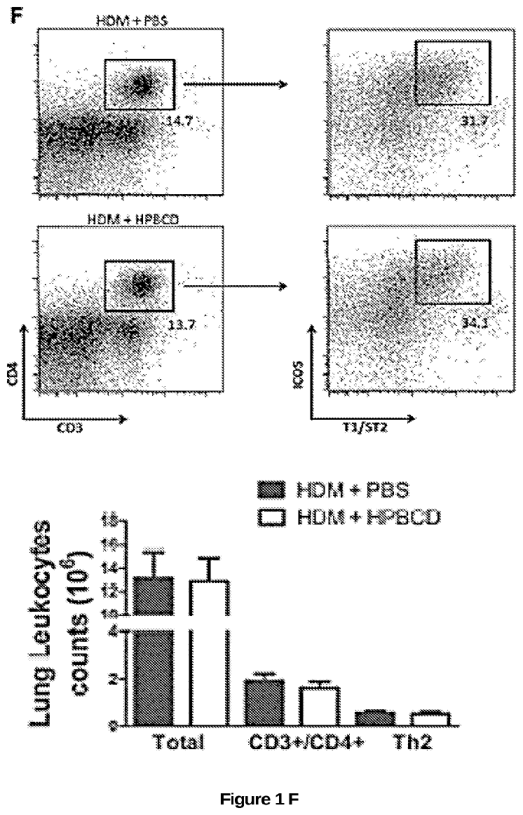

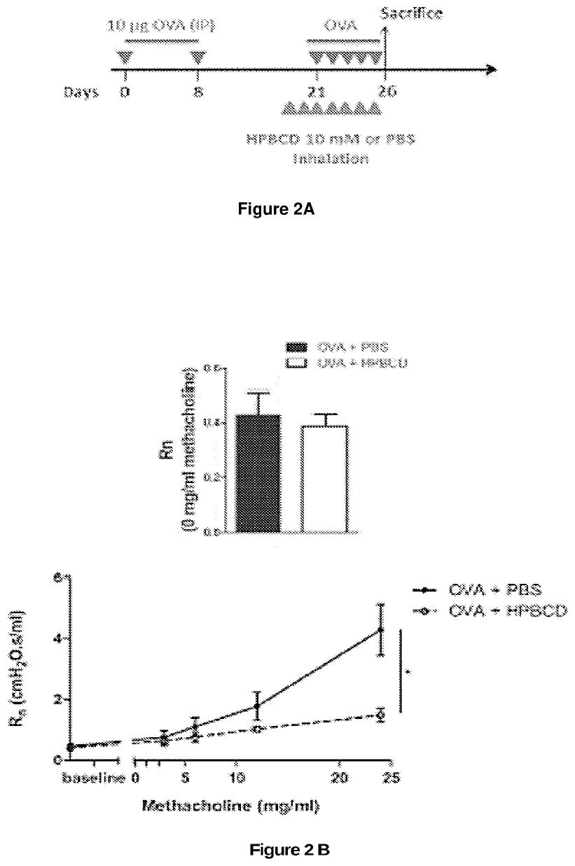

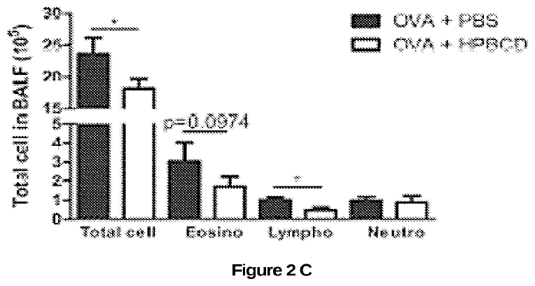

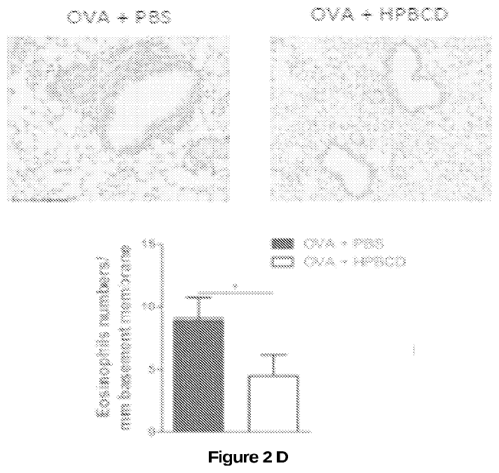

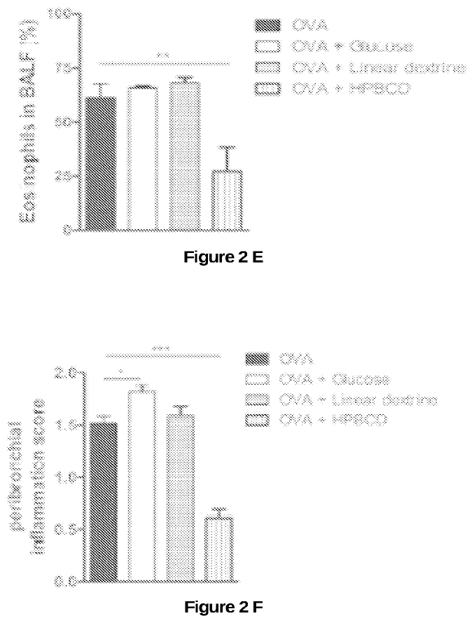

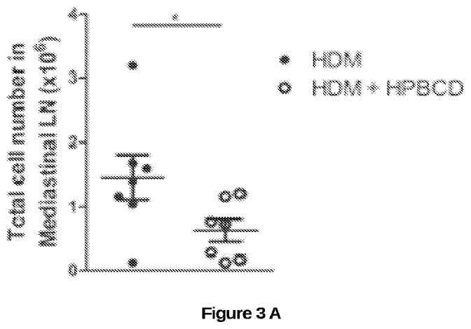

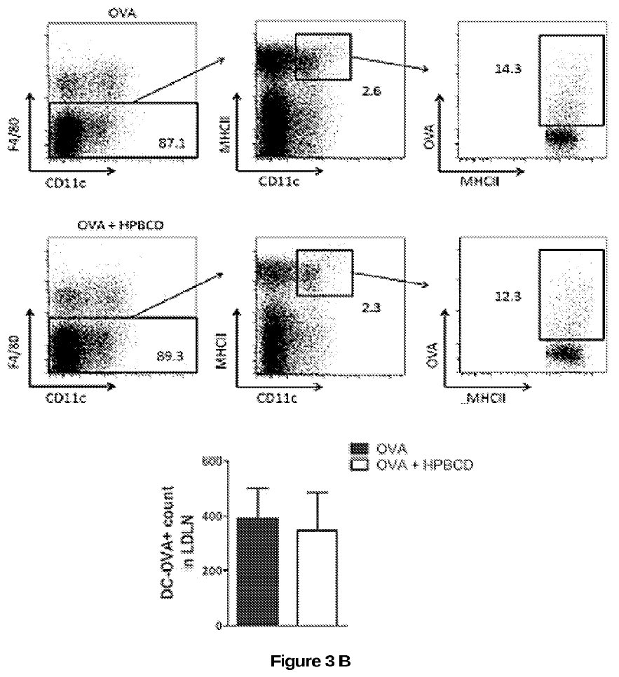

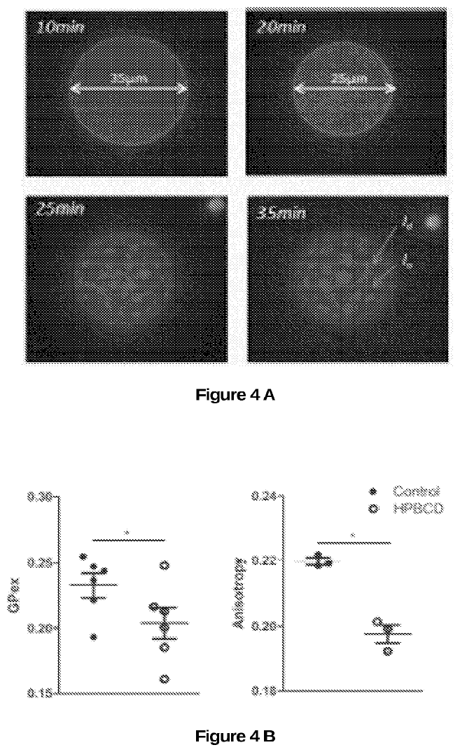

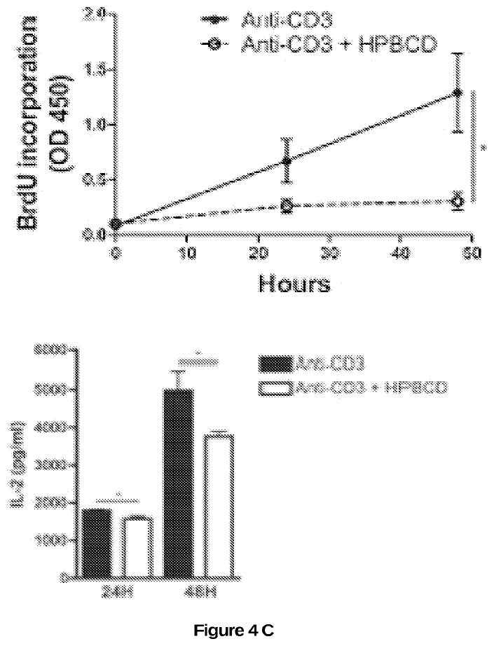

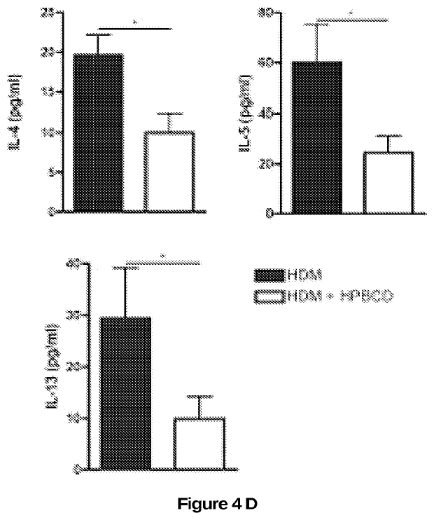

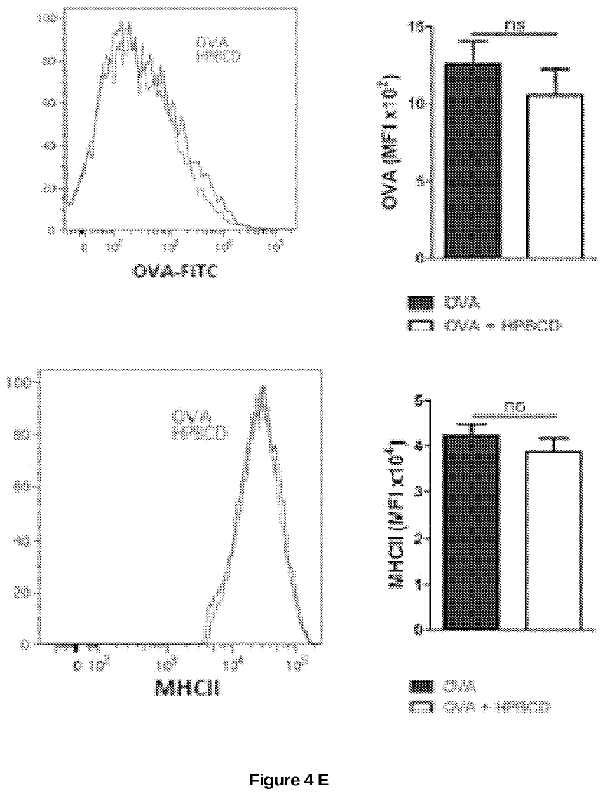

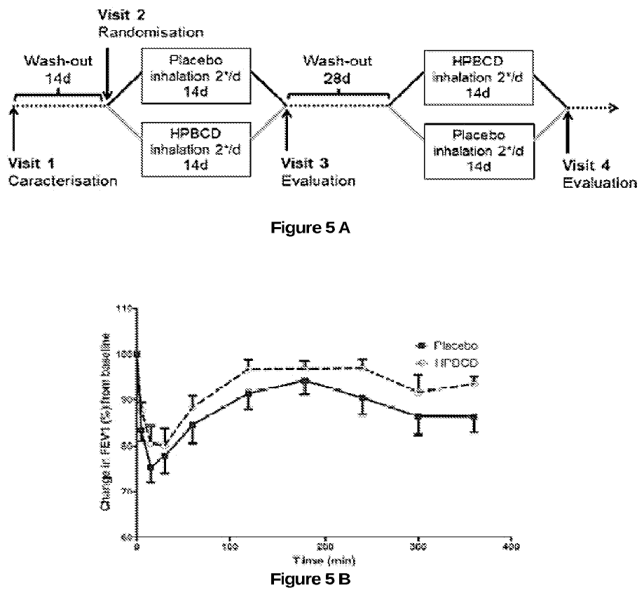

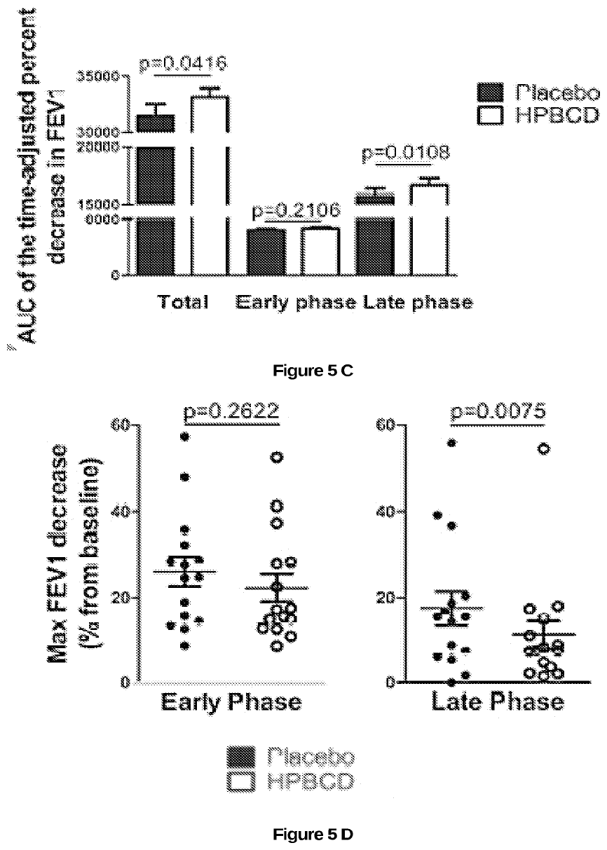

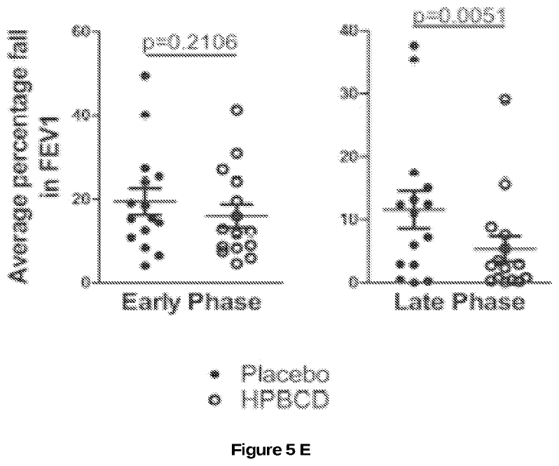

SHORT DESCRIPTION OF THE DRAWINGS

| | |
|---|---|
| FIG. 1 | FIG. 1 shows how HPBCD protects against allergen-induced |
| | inflammation and AHR in mice |
| FIG. 1A | Protocol for HDM-induced inflammation and airway hyper-reactivity, |
| | intranasal 100 mcg/50 mcl |
| FIG. 1B | Airway function tests following the exposure to increasing doses of |
| | methacholine 3-12 mg/ml and baseline lung resistance without |
| | methacholine. |
| FIG. 1C | BALF cell counts eosinophils, lymphocytes, neutrophils |
| FIG. 1D | Haematoxylin and Eosin stained lung sections and inflammation scores |
| FIG. 1E | Alcian blue staining of lung sections |
| FIG. 1F | Flow cytometry analysis of lung TH2 cells and total leukocytes counts |
| FIG. 2 | FIG. 2 shows how HPBCD targets ovalbumin-induced airway |
| | responsiveness and inflammation. |
| FIG. 2A | Protocol for OVA induced inflammation and AHR-inhalation. |
| FIG. 2B | Airway resistances measurement following methacholine challenges with |
| | Increasing doses (3--24 mg/ml) and baseline lung resistance without |
| | methacholine. Rn, Newtonian Resistance. |
| FIG. 2C | Cell content in BALF. Eosino, eosinophils; Lympho, lymphocytes, Neutro, |
| | neutrophils. |
| FIG. 2D | Representative congo red stains of lung sections and number of |
| | eosinophils/mm basement membrane. |
| FIG. 2E | Eosinophil percentage (%) in BALF in animals treated with HPBCD, |
| | linear dextrines and glucose. |
| FIG. 2F | Peribronchial inflammation scores. |
| FIG. 2E-F | n = 7-8mice/group, means +/− s.e.m, one-way ANOVA test, * P < 0.05, ** |
| | P < 0.01, *** P < 0.001, Data are representative of two independent |
| | experiments. |
| FIG. 3 | FIG. 3 shows how HPBCD reduces T-cell proliferation in lund |
| | draining lymph nodes (LDLN). |
| FIG. 3A | Total cell number in LDLN |
| FIG. 3B | Flow cytometry analysis of DC-OVA+ (F4/80− CD11c+ MHCII+) |
| | in LDLN in LDLN. |
| FIG. 3A-B | N = 7 mice/group, means +/− s.e.m, two-tailed Mann-Whitney test, |
| | *P < 0.05, Data are representative of at least two independent |
| | experiments. |
| FIG. 4 | FIG. 4 shows how lipid extraction by HPBCD impairs T-cell |
| | activation and proliferation. |
| FIG. 4A | Phase separation (liquid-ordered (lo)-liquid-disordered (ld)) study in |
| | GUVs (TR-DPPE (red, ld); NBD-PE (green, lo) further to HPBCD (5 mM) |
| | incubation. |
| FIG. 4B | GPex (n = 6) and anisotropy (n = 3) on jurkat cells (HPBCD (5 mM) |
| | incubation 3 h. |
| FIG. 4C | Proliferation study (BrdU incorporation-n = 4) and IL-2 secretion in |
| | culture medium (ELISA-n = 6) on naïve CD4 + T-cells (Balb/c) |
| | stimulated with plate-bound anti-CD3 (3 μg/ml). |
| FIG. 4D | Ex-vivo re-stimulation study of LDLN cells. ELISA measurement of |
| | secreted II-4, −5 and −13. |
| FIG. 4B-D | Two-tailed paired t test. |
| FIG. 4E | Flow cytometry of lung DCs (F4/80− CD11c+ MHCII+) for OVA-FITC |
| | and MHCII expression (MFI) n = 6 mice/group, means +/− s.e.m, |
| | two-tailed Mann-Whitney test. Data are representative of two independent |
| | experiments. |
| FIG. 5 | FIG. 5 shows how inhaled HPBCD allergen-induced |
| | bronchoconstriction in a proof of concept clinical trial |
| FIG. 5A | Clinical trial profile. |
| FIG. 5B | Change in FEV1 (%) from baseline following allergen (HDM) challenge. |
| FIG. 5C | AUC of time-adjusted percent FEV1 decrease. |
| FIG. 5D | Maximum FEV1 decrease (% from baseline) during early (0-60) min and |
| | late (180-360 min) phases (B-E). |
| FIG. 5E | Average percentage fall in FEV1 decrease during early (0-60 min) and |
| | late (180-360 min) phases. |
| FIG. 5B-E | N = 15 mild to moderate asthmatic patients, means +/− s.e.m., one tailed |
| | Wilcoxon matched pairs test. |

### EXAMPLES

The following exemplary embodiments further illustrate the present invention without limiting its scope. FIGS. 1 to 5 illustrate the examples in more detail.

### Abbreviations

APCs Antigen presenting cells
AHR Airway hyper-reactivity
AUC FEV1-versus-time curve
BALF Bronchoalveolar lavage fluid

### BrdU Bromodeoxyuridine

ELISA Enzyme-linked-immuno-sorbent-assay
FEV1 Forced expiratory volume after one second

### GCPs Good Clinical Practices

GUVs Giant unilamellar vesicles
HDM House dust mite
HPBCD Hydroxypropyl-beta-cyclodextrin
i.n. intranasal instillations

### Id Liquid-disordered

LDLN Lung-draining lymph nodes

### lo Liquid-ordered

mcg microgram
mcl microliter
PBS Phosphate-buffered saline
PD20 Provocative dose that causes a 20% fall in FEV1
POPC 1-palmitoyl-2-oleoyl-sn-glycero-3-phosphocholine
Rn Newtonian resistances
SEM Standard error of the mean
TRM Long-term resident memory T-cells

### Example 1: 15 mMol HPBCD Saline Isotonic Solution

Dissolve 21.73 grams Kleptose® HPBCD (HP-Betadex, available from Roquette Freres, France) and 8.54 grams NaCl in 1 Liter water for injection, or sterilize under heat steam.

### Example 2: 15 mMol HPBCD PBS pH7.4 Based Solution

Dissolve 21.73 grams Kleptose® HPBCD (HP-Betadex); 7 g of NaCl; 0.2 g of KCl; 1.44 g of Na2HPO4; 0.24 g of KH2PO4 in 800 ml sterile water. Adjust pH to 7.4 with HCl 0.1N. Adjust volume to 1 L with additional distilled H2O. Sterilize by autoclaving or dispense in sterile flasks using double filtration 5 microns and 0.22 microns.

### Example 3: 5 mMol HPBCD Saline Isotonic Solution

Dissolve 8.75 grams Kleptose® HPBCD® (HP-Betadex) and 8.74 grams NaCl in 1 Liter water for injection, or sterilize under heat steam.

### Example 4: 25 mMol HPBCD PBS pH7.4 Based Solution

Dissolve 36.22 grams Kleptose® HPBCD (HP-Betadex); 6.47 g of NaCl; 0.2 g of KCl; 1.44 g of Na2HPO4; 0.24 g of KH2PO4 in 800 ml sterile water. Adjust pH to 7.4 with HCl 0.1N. Adjust volume to 1 L with additional distilled H2O. Sterilize by autoclaving or dispense in sterile flasks using double filtration 5 microns and 0.22 microns.

### Example 5: 40 mMol HPBCD Saline Isotonic Solution

Dissolve 57.91 grams Kleptose® HPBCD (HP-Betadex) and 7.94 grams NaCl in 1 Liter water for injection, or sterilize under heat steam.

### Example 6: 25 mMol HPBCD Saline Isotonic Solution

Dissolve 36.22 grams Kleptose® HPBCD (HP-Betadex) and 8.74 grams NaCl in 1 Liter water for injection, or sterilize under heat steam.

### Example 7: 10 mMol HPBCD Citrate pH. 4.5 Based Solution

Dissolve 17.45 grams Kleptose® HPBCD (HP-Betadex); 8.27 g of NaCl; 0,306 g Citric acid monohydrate, 0,500 g Sodium citrate dihydrate in 800 ml sterile water. (Adjust pH to 4.5 with HCl 0.1N or NaOH 0.1N if need be). Adjust volume to 1 L with additional distilled H2O. Sterilize by autoclaving or dispense in sterile flasks using double filtration 5 microns and 0.22 microns.

### Example 8: 40 mMol HPBCD Citrate pH. 4.5 Based Solution

Dissolve 57.91 grams Kleptose® HPBCD (HP-Betadex); 7.97 g of NaCl; 0,306 g Citric acid monohydrate, 0,500 g Sodium citrate dihydrate in 800 ml sterile water. (Adjust pH to 4.5 with HCl 0.1N or NaOH 0.1N if need be). Adjust volume to 1 L with additional distilled H2O. Sterilize by autoclaving or dispense in sterile flasks using double filtration 5 microns and 0.22 microns.

### Example 9: 50 mMol HPBCD Saline Isotonic Solution

Dissolve 72.38 grams Kleptose® HPBCD (HP-Betadex) and 7.77 NaCl grams in 1 Liter water for injection, (or sterilize under heat steam)

### Example 10: HPBCD Inhalation Reduces Allergen-Induced Inflammation and Airway Hyperresponsiveness

The impact of HPBCD on asthma-associated inflammation and bronchial hyperresponsiveness was investigated in a mouse model of airway inflammation and hyperresponsiveness.

Six to seven mice per group were tested. Two-tailed Mann-Whitney test were performed with p-values of *P<0.05, **P<0.01 in two independent experiments.

Two intranasal instillations of house dust mite were performed on days 0 and 7 to induce the migration of specific memory CD4+ T-cells into lung parenchyma, followed by a challenge instillation on day 14. Long-term resident memory T-cells in the lung are associated with a low level of proliferation but are highly effective against known allergens. The last house dust mite instillation was preceded by two days of HPBCD or PBS inhalations and followed by three inhalations until sacrifice (FIG. 1A). Airway hyperresponsiveness is a characteristic of asthma and is mainly induced by structural changes and inflammation in the airways. Airway responsiveness was evaluated by exposing animals to increasing doses of inhaled methacholine and by measuring Newtonian resistances representing the resistance of central or conducting airways. The inventors observed that HPBCD inhalation significantly decreased the airway responsiveness to methacholine while baseline responsiveness was similar between groups (FIG. 1B). The bronchoalveolar lavage fluid (BALF) was then recovered and analyzed for cell content. The eosinophilic inflammation related to allergen exposure was significantly decreased in HPBCD-exposed animals as compared to placebo (FIG. 1C). The extent of inflammation around the bronchi (FIG. 1D) and the number of Alcian blue positive mucus producing epithelial cells (FIG. 1E) were also reduced when mice were treated with HPBCD as compared to placebo. The effect of HPBCD inhalation on T-cell numbers in lung parenchyma was investigated by flow cytometry. Neither the number of total CD4+ T-cells (CD3+CD4+) nor the number of TH2 cells (CD3+CD4+T1ST2+ ICOS+) were modified, suggesting the absence of significant T-cell death further to HPBCD inhalations (FIG. 1F). These results demonstrate the effect of HPBCD on allergen-induced inflammation and hyperresponsiveness and were confirmed in another model of allergen-exposure using nebulized ovalbumin (FIG. 2A). In this model, HPBCD inhalations did also significantly decrease the airway hyperresponsiveness induced by ovalbumin (FIG. 2B) and the number of inflammatory cells in BALF as well as the number of eosinophils around the bronchi (FIGS. 2C and 2D).

### Test Protocol of Example 1

Mice

Males BALB/c mice of 6 to 8 weeks old were purchased from Janvier Labs (Saint-Berthevin, France). All mice were bred and housed in University facilities. All experiences and protocols were previously approved by local ethic (animal care and use) committee from the University of Liege.

Reagents and Antibodies

Lyophilized HDM (Dermatophagoides pteronyssinus) extracts for animal studies were purchased from Greer Laboratories (Lenoir, USA). Methacholine and ovalbumin were from Sigma-Aldrich (Karlsruhe, Germany). HPBCD (Kleptose® HPB−molar substitution=0.64) was kindly provided by Roquette (Lestrem, France). D(+)-Glucose and linear dextrin were purchased from VWR (Leuven, Belgium). BrdU colorimetric cell proliferation ELISA was from Roche (Mannheim, Germany). TR-DPPE (Texas red 1,2-dipalmitoyl-sn-glycero-3-phosphoethanolamine) and NBD-PE (N-(7-nitrobenz-2-oxa-1,3-diazol-4-yl)-1,2-dihexadecanoyl-sn-glycero-3-phosphoethanol-amine) were purchased from Invitrogen (Paisley, Scotland). DPH (1,6-diphenyl-1,3,5-hexatriene) and Laurdan (6-dodecanoyl-2-dimethyl-aminonaphtalene) were purchased from Molecular Probes (Invitrogen, Carlsbad, Calif.). POPC (1-palmitoyl-2-oleoyl-sn-glycero-3-phosphocholine), sphingomyelin and cholesterol were ordered from Avanti Polar Lipids (Birmingham, UK). OVA-fluorescein isothiocyanate (FITC) was from Invitrogen (Paisley, Scotland). Phycoerythrin-conjugated anti-F4/80 (BM8), APCcyanine7 conjugated anti-CD11c (N418) and PerCP-Cy5.5 conjugated anti-MHCII (AF6-120.1) were from eBioscience (San Diego, USA). APCcyanine7 conjugated anti-CD3 (17A2) and BV510 conjugated anti-CD4 (RM4-5) were purchased from BD biosciences (Mississauga, Canada). Alexa 647 conjugated anti-ICOS (C398.4A) was obtained from Biolegend (San Diego, USA). PE conjugated anti-T1ST2 (DJ8) was ordered from MD Biosciences (St Paul, USA). Isotype controls and antibodies were from the same manufacturer. 2.4G2 Fc receptor antibodies were produced in house.

Flow Cytometry

Staining reactions were performed at 4° C. Cells were previously incubated with 2.4G2 Fc receptor antibodies to reduce nonspecific binding. Data were analyzed using FlowJo software.

Airway Inflammation Protocol

Mice were lightly isoflurane-anesthetized and received an intra-nasal (i.n) instillation of HDM extract (100 μg; 50 μl) in endotoxin-free saline on days 0, 7 and 14. Mice were subjected to inhalation (generated by ultrasonic nebulizer (Devilbiss 2000)) of HPBCD 10 mM or endotoxin-free saline for 40 minutes between day 12 and 16. On day 14, they received the inhalation 1 h before the last i.n. instillation of HDM. After measurement of bronchial responsiveness using the FlexiVent System, mice were sacrificed on day 17. Mice submitted to ovalbumin as allergen were i.p injected with 10 mcg ovalbumin grade 5 and exposed to ovalbumin grade 3 by inhalation between day 21 and 25. Mice were treated with HPBCD 10 mM given by inhalation between day 19 and 25. After measurement of bronchial responsiveness using the FlexiVent System, mice were sacrificed on day 26.

Measurements of Bronchial Responsiveness

Mice were anesthetized by intraperitoneal injection with a mixture of ketamine (10 mg/ml, Merial, Brussel, Belgium) and xylazine (1 mg/ml, VMD, Arendonk, Belgium). A tracheotomy was performed by insertion of a 20 gauge polyethylene catheter into the trachea. Mice were ventilated with a flexiVent small animal Ventilator® (SCIREQ, Montreal, Canada) as previously described (18). Respiratory parameters following methacholine challenge (3, 6, and 12 gr/l (or 24 gr/l)) were evaluated using a 3 sec broadband signal to measure input impedance from 1 to 20.5 Hz and to calculate constant-phase model parameters (Quick-prime 3). Newtonian resistance (Rn) was the main parameter measured during the challenge.

Bronchoalveolar Lavage Fluid (BALF)

Mice were sacrificed and a bronchoalveolar lavage was performed using PBS-EDTA 0.05 mM (Calbiochem, Darmstadt, Germany). Cells were recovered by gentle manual aspiration. After centrifugation of bronchoalveolar fluid (BALF) (1200 rpm; 10 min; 4° C.), the cell pellet was resuspended in 0.5 ml PBS-EDTA 0.05 mM. The differential cell counts were performed on cytocentrifuged preparations (Cytospin) after Diff-Quick (Dade, Belgium) staining.

Lung Histology and Tissue Processing

Left lung was excised and snap frozen in liquid nitrogen. Right lung was infused with 4% paraformaldehyde, embedded in paraffin and used for histology. Sections of 5 μm thickness were cut off from paraffin and were stained with haematoxylin-eosin to estimate the extent of inflammation (18) and with Alcian blue (mucin stain).

Lung TH2 Cells Measurement

To obtain single-lung-cell suspensions, lungs were perfused with 10 ml HBSS through the right ventricle, razor-cut into small pieces and digested for 1 hour at 37° C. in 1 mg/ml collagenase A (Roche) and 0.05 mg/ml DNasel (Roche) in HBSS. Leukocytes were enriched thanks to Percoll gradient (Easycoll, Millipore). TH2 cells were defined as CD3+CD4+T1ST2+ ICOS+ cells. Flow cytometry was performed on a FACScanto II (Becton Dickinson, Mountain View, Calif.).

Allergen Uptake and DCs Migration

To assess lung DCs (F4/80-CD11c+ MHCII+) allergen uptake and DCs migration to LDLN, mice were injected i.n. with 100 μg OVA-FITC. 24 h later, lungs and LDLN were analyzed by flow cytometry for the presence of antigen-loaded DCs (FITC+ DCs). To evaluate the impact of HPBCD on allergen uptake and DCs migrations, mice were treated with HPBCD inhalation on D-2; D-1 and 1 h before i.n. instillation of OVA-FITC. Flow cytometry was performed on a FACScanto II (Becton Dickinson, Mountain View, Calif.).

### Example 11: Inhaled HPBCD Reduces Allergen-Induced Bronchoconstriction in Human Asthmatics

Seventeen mild to moderate asthmatics sensitized to HDM were included in a double blind crossover study comprising allergen challenges with HDM. Surprisingly, patients displayed a lower decrease in FEV1 after HDM challenge (area under the FEV1-time curve (AUC) 0-360 min) when treated with HPBCD inhalations as compared to the placebo treatment periods (FIG. 5B). The analysis (AUC) of FEV1 decrease during early and late phases following HDM challenge shows a predominant effect of HPBCD inhalation on the prevention of FEV1 decrease during the late phase reaction (FIG. 5C). Maximum FEV1 decrease and average percentage FEV1 decrease after HDM challenge were calculated and found to be significantly lower in HPBCD-treated patients (FIGS. 5D and 5E). These experiments show a significant effect of HPBCD inhalation on the prevention of allergen-induced FEV1 decrease during the late phase. No significant differences between HPBCD and placebo were seen during the early bronchoconstriction phase. No significant changes were observed in hematology, serum biochemistry, urinalysis, vital signs, ECG tests and chest X-ray between treatment groups

### Test Protocol of Example 2

Clinical Trials Procedures

Clinical trial protocols have been approved by an independent ethical committee (CHU Liege—University of Liege) in accordance with GCPs, Declaration of Helsinki and European regulations (EudraCT). All participants gave written informed consent before any study-specific procedure. For the phase 1 clinical trial, 8 healthy subjects (4 women and 4 men) from 18 to 40 years were recruited and enrolled in the study at the University Hospital of Liege (Belgium). Patients were randomized and received first the placebo (NaCl 0.9%) or HPBCD at 2.5 mM and 8 days after, the opposite treatment. After 1 week, all patients received HPBCD at 15 mM. One month later, all patients received HPBCD at 15 mM during 5 consecutive days. Apyrogenic and sterile HPBCD powder was diluted with NaCl 0.9% before inhalation (8 ml) with an ultrasonic nebulizer (Devilbiss 2000). General symptoms, asthma scores (ACQ), dyspnea, chest Rx, clinical biology, ECG, vital signs, spirometry, NO, sputum (induced by NaCl 4.5%) were analyzed after treatment periods. The second clinical trial (phase 2a-proof of concept) was a double-blinded, cross-over, placebo-controlled study. 17 mild to moderate asthmatic patients were included in the study at two different sites (University Hospital of Liege and University Hospital Erasme) in Belgium. Characterization visits consisted of dyspnea symptoms recording, ECG, chest Rx, clinical biology, spirometry and vital signs analysis. PD20 (provocative dose that causes a 20% fall in FEV1 from the saline alone value) to inhaled allergen was determined. In order to determine the individual bronchial responsiveness to the allergen, a challenge was conducted with a Dermatophagoides pteronyssinus extract (Stallergen; Antony, France) diluted in an isotonic saline solution that had a reactivity index ranging from 0.2 to 5. The PD20 of the allergen was calculated from a cumulative dose-response curve, as described previously, Cataldo et al., Matrix metalloproteinase-9, but not tissue inhibitor of matrix metalloproteinase-1, increases in the sputum from allergic asthmatic patients after allergen challenge, Chest. 2002; 122(5):1553-9. All healthy subjects received a cumulative concentration of Dermatophagoides pteronyssinus with a reactivity index of 6. After 14 days wash-out period, patients were randomly divided into two groups and received either HPBCD 15 mM or Placebo (NaCl 0.9%) by inhalation (ultrasonic nebulizer (Devilbiss 2000)) twice daily. At the end of this treatment period, lung function (FEV1) was analyzed after allergen (HDM) challenge under the supervision of experienced respiratory clinical physiologists. FEV1 was measured after allergen inhalation challenge at 5, 15, 30, 60, 120, 180, 240, 300 and 360 min. After 28 days wash-out period, patients received the opposite treatment for another 14 days and the FEV1 following allergen challenge was evaluated. All clinical parameters were examined after each treatment period. The endpoint of this proof of concept study was changes in FEV1 over 0-360 min post-allergen challenges. FEV1 in early phase (0-60 min) and late phase (180-360 min) were expressed as: area under the FEV1-versus-time curve (AUC), maximum percentage fall and average percentage fall (calculated by dividing the area under the curve of the percentage fall in FEV1 by the duration of the response period). One patient who received placebo had negative maximum FEV1 decrease and negative average percentage fall in FEV1. These negative values were truncated to 0. Patients had access to 132-agonist (salbutamol 100 μg) throughout the study as an «as needed» treatment.

Preparation and Visualization of GUVs

GUVs were prepared by electro-formation. Briefly, 1 mcl of a chloroform solution of SM/Chol/POPC (1:1:1) was spread on an indium tin oxide-covered glass. The fluorescent probes (TR-DPPE and NBD-PE) were added to the chloroform solution at a concentration of 0.1% mol/mol. The solution was dried in a vacuum chamber for 2 h. An electro-formation chamber was constructed using another indium tin oxide-covered glass slide, with the conducting face pointed toward the interior of the electro-formation chamber, which was filled with a 0.1 M saccharose solution. Polydimethylsiloxane containing 5% fumed silica was used to separate the two glass slides. The GUVs were grown by applying a sinusoidal alternating current of 10 Hz and 1 V for 2 h at 60° C. GUVs were analyzed on Axioskop 40 microscope (Carl Zeiss, Jena, Germany) with a 40×/0.75 Zeiss EC Plan-Neofluar® objective and the images were recorded with a Nikon digital sight DS-5 M camera (Nikon, Tokyo, Japan). TR-DPPE was excited at 561 nm and analyzed at 617 nm. NBD-PE was excited at 460 nm and analyzed at 535 nm. Phase separation (Id/lo) was evaluated further to incubation with HPBCD at 5 mM.

T-Cell Membrane Organization and Rigidity

To analyze the impact of HPBCD on mobility and polarity of phospholipids at the glycerol backbone level, Jurkat cells were incubated with Laurdan. Briefly, 1×106 cells were seeded in 3 ml RPMI 10% FBS 1 pen/strep and treated at 37° C. with HPBCD (5 mM). After 3 h, medium was replaced and cells were incubated with 1.4 mcM Laurdan for 1 h at 37° C. GPex was determined at 37° C. as a measure of membrane organization and rigidity as described in Lorent et al., Induction of Highly Curved Structures in Relation to Membrane Permeabilization and Budding by the Triterpenoid Saponins, α- and δ-Hederin, The Journal of Biological Chemistry 2013; 288(20):14000-17. The lipid dynamic/fluidity at the acyl chain further to HPBCD incubation was analyzed in the same way thanks to DPH probe. Anisotropy was determined at 37° C. as a measure of membrane rigidity.

In-Vitro T-Cell Proliferation Assay

HPBCD was used at a concentration of 5 mM. This concentration was not associated with cytotoxicity (as measured after 48 h incubation by BrdU incorporation). Naïve CD4+ T cells were isolated from Balb/c mice spleen and purified with a MACS® negative selection kit. 1.5×105 cells were seeded in a 96 well plate and incubated with HPBCD 5 mM (in RPMI 10% FBS 1% pen/strep) or with medium alone for 3 h. Cells were then recovered and plated in an anti-CD3 (3 mcg/ml) coated well for 24 or 48 h with HPBCD (5 mM) or with culture medium alone at 37° C. 5% CO2. During the last 2 h of the proliferation test, BrdU was added to medium and incorporation was quantified by ELISA following manufacturer instructions. Other wells were used to assess IL-2 secretion (ELISA) in the medium after 24 and 48 h of anti-CD3 stimulation.

In-Vitro T-Cell Stimulation Assay

Cells from lung draining lymph nodes were isolated from mice previously i.n challenged with HDM. These cells were re-stimulated in 96 wells plate with 30 mcg HDM in RPMI. Supernatants were assessed after 48 h for IL-4, IL-5 and IL-13 secretion by ELISA.

Statistical Analysis

Phase 2 Clinical Trial—Inclusion and Exclusion

Inclusion Criteria
Male or female suffering from mild to moderate asthma
Age: 18765 years
No current smokers (max tobacco consumption: 10/year)
Sensitization to house dust mite (Dermatophagoïdes Pteronyssinus) controlled by RAST or prick tests
No regular therapy for asthma (exception: short acting bronchodilators)
No asthma exacerbation or respiratory tract infection during the 6 weeks preceding the study inclusion
Body mass index (BMI) 18-28 kg/m²
No significant concomitant disease or vital signs abnormalities
Informed consent to be given
Subject available during the study

Exclusion Criteria
Any medication taken in the last 28 days
Active smokers or addicted to any other drug
Drug allergy
Alcohol (>2 glasses/day) and coffee (>4 cups/day) consumption
Medical history of hearth, kidney, liver problems that could interfere with the study
Concomitant participation to another study
No informed consent

Data are presented as mean±SEM unless specified. The differences between mean values were estimated using a two-tailed Mann-Whitney test (animal and in vitro experiments) unless specified. All animal experiments were repeated at least 2 times; n 6 in each experimental group. ANOVA Friedman test (single dose) and Wilcoxon signed rank test (multiple doses) were used to analyze results obtained during the phase 1 clinical trial. One-tailed Wilcoxon matched pairs test was used to evaluate intra-individual differences in the proof of concept clinical trial. A P value less than 0.05 was considered significant. GraphPad Prism and Statistica softwares were used to analyze results.

The results are shown in following Table 1:

TABLE 1
Phase 1 clinical trial NO, FEV1 and eosinophils (sputum)
were followed after single (HPBCD 2.5 mM and 15 mM)
or multiple (HPBCD 15 mM/5 days) administration(s). QT and
QTc were assessed during the multiple dose trial. n = 8 healthy
subjects, means +/−-s.d. ANOVA Friedman test (single dose)
and Wilcoxon signed rank test (multiple dose).

| Parameters | | | | |
|---|---|---|---|---|
| | Placebo | HPBCD 2.5 mM | HPBCD 15 mM | |
| Single dose | mean ± SD | mean ± SD | mean ± SD | P-Value |
| No measurements | 22.57 ± 8.07 | 23.85 ± 7.56 | 28.48 ± 17.92 | 0.69 |
| (ppb) 15 min after inhalation | | | | |
| FEV1 (l/sec) 15 min | 4.04 ± 0.87 | 4.08 ± 0.87 | 4.01 ± 0.87 | 0.22 |
| after inhalation | | | | |
| FEV1 (l/sec) 1 h | 4.06 ± 0.82 | 4.11 ± 0.83 | 4.04 ± 0.86 | 0.20 |
| after inhalation | | | | |
| FEV1 (l/sec) 3 h | 4.09 ± 0.83 | 4.10 ± 0.82 | 4.07 ± 0.85 | 0.66 |
| after inhalation | | | | |
| FEV1 (l/sec) 5 h | 4.15 ± 0.88 | 4.11 ± 0.87 | 4.08 ± 0.92 | 0.66 |
| after inhalation | | | | |
| Eosinophils (%) 5 h | 0.10 ± 0.21 | 0.00 ± 0.00 | 0.20 ± 0.49 | 0.66 |
| after inhalation | | | | |

| Multiple dose | Before first | After 5 days | |
|---|---|---|---|
| (HPBCD 15 | inhalation | inhalation | |
| mM) | mean ± SD | mean ± SD | |
| No measurements (ppb) | 25.74 ± 11.13 | 25.16 ± 9.76 | 0.78 |
| 15 min after inhalation | | | |
| FEV1 (l/sec) 15 min | 4.10 ± 0.84 | 4.13 ± 0.87 | 0.31 |
| after inhalation | | | |
| FEV1 (l/sec) 1 h | 4.10 ± 0.84 | 4.07 ± 0.82 | 0.26 |
| after inhalation | | | |
| FEV1 (l/sec) 3 h | 4.10 ± 0.84 | 4.11 ± 0.87 | 0.83 |
| after inhalation | | | |
| FEV1 (l/sec) 5 h | 4.10 ± 0.84 | 4.10 ± 0.87 | 0.67 |
| after inhalation | | | |
| Eosinophils (%) 5 h | 0.00 | 0.00 | — |
| after inhalation | | | |
| QT (ms) | 384.80 | 393.50 | 0.23 |
| QtTc (ms) | 408.00 | 393.30 | 0.14 |

1. A method of treatment of T-cell dysfunction in pulmonary tissue, wherein cyclodextrin is administered per inhalation in an amount effective to reduce membrane order in the cells in subjects with T-cell dysfunction in their pulmonary tissue, preferably without causing treatment limiting side effects, such as those selected from the group consisting of renal clearance, hepatic impairment as expressed by elevated levels of transaminase, and wheezing after administration, as compared to subjects untreated with the cyclodextrin.

2. The method of claim 1, wherein the T-cell dysfunction in pulmonary tissue is asthma-induced.

3. The method of claim 1, wherein the cyclodextrin is Hydroxypropyl-beta-cyclodextrin.

4. The method of claim 1, wherein the cyclodextrin is an inhalable aqueous solution.

5. The method of claim 4, wherein the cyclodextrin concentration is from 5 millimolar to 50 millimolar.

6. The method of claim 4, wherein the cyclodextrin concentration is from 7 millimolar to 40 millimolar.

7. The method of claim 4, wherein the cyclodextrin concentration is from 10 millimolar to 30 millimolar.

8. The method of claim 1, wherein the cyclodextrin is a spray-dried powder.

9. The method of claim 1 in pulmonary tissue, wherein the cyclodextrin is administered in an amount effective to reduce membrane order in cells.

10. The method of claim 1, wherein the cyclodextrin is administered per inhalation in the amount of 0.1 mg to 30 mg per day.

11. The method of claim 1, wherein the cyclodextrin is administered per inhalation in the amount of 0.5 mg to 20 mg per day.

12. The method of claim 1, wherein the cyclodextrin is administered per inhalation in the amount of 1 mg to 10 mg per day.

13. The method of claim 1, wherein the cyclodextrin is administered to children aged up to two years per inhalation in the amount of 0.1 mg to 0.5 mg per day.

14. The method of claim 1, wherein the cyclodextrin is administered to children aged from two to 6 years per inhalation in the amount of 0.5 mg to 1 mg per day.

15. The method of claim 1, wherein the cyclodextrin is administered to children aged from 6 years to 14 years per inhalation in the amount of 1 mg to 2 mg per day.

16. The method of claim 1, wherein the cyclodextrin is administered from 0.1 mg to 15 mg per day in mild to moderate allergen-induced asthma and from 1 mg to 30 mg in severe allergen-induced asthma.
